(19) United States
(12) Patent Application Publication
(10) Pub. No.: US 2003/0153790 A1
(43) Pub. Date: Aug. 14, 2003

(54) Process for the preparation of 1-(2,6,6-trimethyl-2-cyclohexene-1-yl)-but-2-ene-1-one

(76) Inventors: Kudli Shrinivas Prashanth Kumar, Karnataka (IN); Nagaraj Surekha, Karnataka (IN)

Correspondence Address: Drinker Biddle & Reath LLP, One Logan Square, 18th and Cherry Streets, Philadelphia, PA, 19103-6996, US

(21) Appl. No.: 10/074,560

(22) Filed: Feb. 13, 2002

(51) Int. Cl. C07C 45/46

(52) U.S. Cl. 568/356

(57) ABSTRACT

The present invention relates to a novel process for the preparation of 1-(2,6,6-trimethyl-2-cyclohexene-1-yl)-2-but-2-ene-1-one commonly known as α-Damascone, by acylation of unsaturated cyclic hydrocarbon 2,6,6-trimethyl-1-cyclohexene followed by work up and its purification by vacuum distillation.

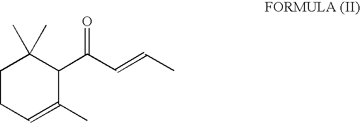

### FIELD OF THE INVENTION

The present invention relates to a novel process for the preparation of 1-(2,6,6-trimethyl-2-cyclohexene-1-yl)-but-2-ene-1-one (II) commonly known as α-Damascone, by acylation of unsaturated cyclic hydrocarbon 2,6,6-trimethyl-1-cyclohexene (I).

### BACKGROUND AND PRIOR ART REFERENCES

Ever since the isolation of α-Damascone (II) (Tetrahedron letters-1973-381 & Agri.Biol.Chem-1974-38-1351) as an important constituent of Black tea aroma was made, its chemical synthesis received much attention. Several synthetic routes have been reported but each differing in the process of manufacture.

A Swiss patent (560663-1975) reported multi-step synthesis of α-Damascone from α-Ionone.

Hung & Hsing-Jang (Tetrahedron letters −1976-46-4129, Can. J. Chem.—1978-56(10)-1368) reported the synthesis of α-Damascone from ethyl ester of 4-keto-α-cyclo geranic acid and obtained a yield of 15-20%.

Kasano & Masanobu (Kinki Daigaku Rikogakubu Kenkyu Hokoku—1978-13-37) reported the synthesis of α-Damascone from Citral as starting material. The process involved cyclisation of Citral to Cyclocitral and thereafter reaction of Cyclocitral with allyl magnesium halide followed by oxidation to α-Damascone.

Snowden (Tetrahedron letter—1982-23(3)-335) reported the synthesis of α-Damascone using Grignard reagent. This process involved reaction between allyl magnesium halide with α-methyl cyclo geranate, followed by fragmentation of potassium dihomhallyic alkoxide under thermolytic conditions.

A European patent (EP 70995-1883) reported four-step synthesis of α-Damascone from an unsaturated acyclic ketone by Wittig & cyclisation reactions.

Visser (Recl. J.R.Neth. Chem. Soc—1983-102(6)-307), reported the synthesis of α-Damascone by a photochemical route.

Mandai (J Org Chem.—1984-49(18)-3403), reported a seven-step synthesis of α-Damascone from γ-Geraniol.

A European Patent (EP 224897-1987) reported the synthesis of α-Damascone from alkenylmagnesium halides with cyclic ketone.

Zaidlewicz (Tetrahedron letter—1986-27(42)-5135) reported six-step synthesis of α-Damascone using allylic organoborane route.

Muller & Bernard L. (Helv. Chim. Acta.—1987-70(7)-1858), reported the synthesis of α-Damascone by bis homoallylic alcohol route and reported a yield of 35% based on the starting material i.e., α-methyl cyclo geranic acid.

Fehr, Charles. (J. Am. Chem. Soc.—1988-110(20)-6909) reported the synthesis of α-Damascone from tandem Grignard reaction with Cyclocitral.

A European Patent (EP326869-1989) reported the synthesis of α-Damascone from Methyl α-cyclogeranate. The process involved usage of expensive Butyl lithium & dimethyl phenyl imidazolidone.

Azzari & Elisabetta. (J. Org Chem.—1990-55(3)-11060 reported a five-step synthesis of α-Damascone from α-Ionone in five steps with a yield of 20% based on α-Ionone.

A Japanese patent (JP4330033-1992) reported the synthesis of α-Damascone from the oxidation of α-Damascol, which is in turn prepared from Cyclocitral

A Japanese patent (JP4279536-1992) reported multi-step synthesis of α-Damascone from 2,4,4-trimethyl-2-cyclohexen-1-ol, which used expensive materials viz., tri butyl tin iodide, potassium hydride, butyl lithium, oxalyl chloride etc.

Mori & Kenji. (Tetrahedron—1993-49(9) 1871) reported eight-step synthesis of α-Damascone from 2,4,4-trimethyl-2-cyclohexene-1-ol and reported a yield of 23%.

Zheng (Chin. Chem. Letter—1992-3(3)-177), reported the synthesis of α-Damascone from allylation of α-Cyclocitral with a yield of 51%.

A Chinese patent (CN1172099-1998) reported the synthesis of α-Damascone from Cyclocitral using allyl zinc or allyl tin reagents.

Naef (Tetrahedron—1986-42-3245), reported the synthesis of α-Damascone from Grignard & Hydride Addition to a ketene intermediate

Luis (J. Org. Chem.—1992-57-2757), reported the synthesis of α-Damascone from Alpha-Ionone in four steps and obtained a yield of 59% based on α-Ionone. The process involved epoxidation, borohydride reduction, sonication of the intermediate bromide and oxidation.

However the applicants have now devised a simple, economical, eco-friendly and high yielding process, which avoids all the difficulties and restrictions of the prior art processes.

### OBJECTS OF THE INVENTION

The primary object of the present invention is to provide a novel process for the preparation of 1-(2,6,6-trimethyl-2-cyclohexene-1-yl)-but-2-ene-1-one (II) commonly known as α-Damascone, by acylation of unsaturated cyclic hydrocarbon 2,6,6-trimethyl-1-cyclohexene (I).

Another object of the present invention is to provide one pot efficient process for the preparation of α-Damascone.

Yet another object of the present invention is to provide a starting material, which is commercially available and can also be produced by known methods.

Still another object of the present invention is to provide a synthetic route for the preparation of α-Damascone by using crotonic anhydride, in high yield.

Yet another object of the present invention is to provide α-Damascone as a synthetic source for flavor and aroma.

Still another object of the present invention is to provide a synthetic route for the preparation of α-Damascone that obviates the difficulties encountered in the prior art references.

### SUMMARY OF THE INVENTION

The present invention relates to a process for the preparation of 1-(2,6,6-trimethyl-2-cyclohexene-1-yl)-but-2-ene-1-one commonly known as α-Damascone, by the acylation of 2,6,6-trimethyl-1-cyclohexene with Crotonic anhydride in the presence of Lewis acid to obtain the product.

### DETAILED DESCRIPTION

Accordingly, the present invention provides a process for the preparation of α-Damascone [1-(2,6,6-trimethyl-2-cyclohexene-1-yl)-2-buten-1-one] represented by formula (II)

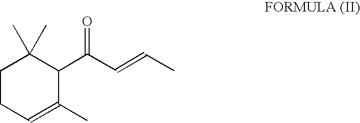

said process comprising the steps of:

(a) adding 2,6,6-trimethyl-1-cyclohexene over a period of 30-95 minutes to a refluxing mixture of an hydrocarbon solvent, Lewis acid and Crotonic anhydride at a temperature in the range of 60-120° C.;

(b) cooling the reaction mixture of step (a) to an ambient temperature of 25-30° C.;

(c) pouring the cooled reaction mixture of step (b) into aqueous alkali carbonate solution and stirring the reaction mixture for about 5-20 minutes;

(d) separating the organic layer and washing the organic layer of step (c) with water till the washing becomes neutral;

(e) drying and removing organic solvent from the organic layer to obtain crude product and purifying the crude product to obtain pure α-Damascone of formula (II).

An embodiment of the present invention, wherein the hydrocarbon solvent is selected from the group consisting of n-hexane, n-heptane, cyclopentane or cyclohexane and most preferably cyclohexane or n-heptane.

Another embodiment of the present invention, wherein the Lewis acid is selected from the group consisting of aluminum chloride, aluminiumbromide, zinc chloride, zinc bromide or borontrifluoride etherate and most preferably zinc chloride.

Yet another embodiment of the present invention, wherein the alkali carbonate is selected from the group consisting of sodium bicarbonate, potassium bicarbonate, sodium carbonate or potassiumcarbonate and most preferably sodium carbonate.

Further embodiment of the present invention, wherein the strength of aqueous alkali carbonate solution is about 5%.

Yet another embodiment of the present invention, wherein the drying of organic layer is done by passing over anhydrous sodium sulphate.

Still another embodiment of the present invention, wherein the crude product is subjected to fractional distillation at a temperature of about 90° C. and at a pressure of about 5 mm Hg.

Yet another embodiment of the present invention, wherein the crude product is purified by way of vacuum distillation.

Still another embodiment of the present invention, wherein the product α-Damascone is obtained in the yield of 70%-85% having a purity of over 95% (GLC).

Yet another embodiment of the present invention, wherein the acylation reaction can also be performed in the absence of solvent at about 20-60° C.

Further embodiment of the present invention, wherein the weight ratio of the organic solvent:2,6,6-Trimethyl-1-Cyclohexene is about 1:3 to obtain higher yields, preferably 1.2-1.5 times the weight of starting material (I).

The Schematic representation of the reaction is provided below in Figure A:

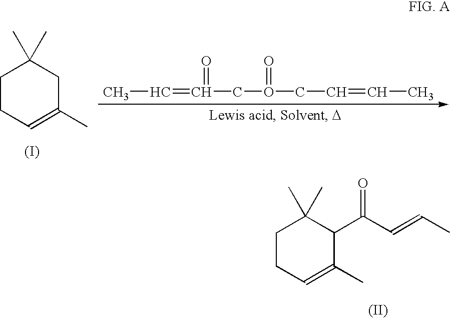

2,6,6-Trimethyl-1-Cyclohexene

The present invention is further explained in the form of preferred embodiments:

As can be seen in FIG. A, the starting material 2,6,6-Trimethyl-1-cyclohexene (I) undergoes acylation with commercially available Crotonic anhydride in the presence of a Lewis acid viz. ZnCl₂, ZnBr₂, AlCl₃ etc, with or without a solvent to give the product (II).

The acylation of the unsaturated hydrocarbon (I) proceeds with shifting of the double bond in the cyclohexane ring.

The acylation reaction is found to proceed with a high conversion in Cyclohexane or n-heptane as the solvent, at a temperature range of 80-100° C. than in other solvents viz. Petroleum ether, Methyl cyclohexane, Chloroform etc. The acylation reaction also proceeds without a solvent at temperatures between 20-60° C., but with lower yields.

In case of solvents, both Cyclohexane and n-heptane are found to give good conversions in dilution levels of 1-3 times by wt of the starting material (I). Lewis acids are found to be equally active at 20-40% by weight of the starting material (I).

Crotonic anhydride is found to give good conversion of the product (II) in level of 1-3 times by the wt of the starting material (I) and the preferred quantity is 1.5-1.8 times the weight of the starting material (I). By the preferred methods of synthesis, applicants are able to get an overall yield of 70-85% of the product (II) by wt based on the starting material and the product is found to be a single isomer of the purity of 95% and above.

The starting material (I) and Crotonic anhydride are known compounds which are commercially available or they are known in literature and can be easily produced according to known methods. The compound (I) can be prepared from commercially available Isophorone ie.3,5,5-Trimethyl-cyclohex-2 and 3-en-1-one by known reduction methods.

Crotonic anhydride can be prepared by commercially available Crotonic acid by acetic anhydride treatment.

It is also observed during the course of the invention that the moisture in the reaction mixture is found to affect the progress of the reaction drastically. Moisture levels upto 1% based which may come from solvent, has not affected the yields of α-Damascone. However, a systematic study of moisture levels of 2% and above has been studied. It has been inferred from the study that moisture levels above 2% drastically reduce the yields of α-Damascone, more particularly when the moisture levels are at 5% and above.

In view of the above, in the present process, all the necessary steps are taken to ensure that moisture levels are controlled by adopting the use of dry solvents.

The present invention is further illustrated in the form following Examples. However, the following examples should not be construed as limiting the scope of the invention.

### EXAMPLE 1

Preparation of 1-(2,6,6-trimethyl-2-cyclohexene-1-yl)-but-2-ene-1-one from 2,6,6-trimethyl-1-cyclohexene

To a stirred suspension of cyclohexane (100 ml), Zinc chloride (20 gms) and Crotonic anhydride (150 gms), 2,6,6-trimethyl-1-cyclohexene (100 ml) is added over a period of 45 minutes at 80° C., after the completion of addition, the mixture is cooled to room temperature and then sodium carbonate aqueous solution (500 ml of 5%) is poured into the mixture. The Cyclohexane layer containing the product (II) is then separated and washed with water to make the layer neutral. Drying the organic layer over sodium sulphate, followed by filtration and distillation under atmospheric pressure to obtain the crude product which is further fractionally distilled at about 90° C. and under a reduce pressure of 5 mm Hg to get 1-(2,6,6-trimethyl-2-cyclohexene-1-yl)-but-2-ene-1-one (II) commonly known as α-Damascone, in the yield of 70 gms and having a purity of 95% (GLC).

BP=253-255° C.(760 mm), 80° C. nD²⁷=1.4884, d²⁷=0.9229

MS: 41, 55, 69, 91, 107, 123, 177, 192

FTIR: 3034, 2954, 2936, 2917, 2863, 1694, 1670, 1628, 1443, 1291, 1082, 971, 919

UV (Max): 221 nm

¹³C NMR(100 M Hz): 201.7, 142.7, 136.2, 129.8, 128.7, 77.5, 77.2, 76.9, 51.7, 34.5, 31.9, 30.0, 29.6, 24.1, 22.6, 18.3

1HNMR(400 MHz): 0.83(s,3H), 0.92(s,3H), 1.48(dd,J₁=3.5 Hz,J₂=2.0 Hz,3H), 1.80(dd,J₁=6.7 Hz,J₂=1.4 Hz,3H), 3.3(m,1H), 5.3(m,1H), 6.1(dq,J₁=15.5 Hz,J₂=1.4 Hz,1H), 6.8(dq,J₁=15.5 Hz,J₂=6.7 Hz,1H)

### EXAMPLE 2

### Preparation of 1-(2,6,6-trimethyl-2-cyclohexene-1-yl)-but-2-ene-1-one from 2,6,6-trimethyl-1-cyclohexene

To a stirred suspension of n-heptane (100 ml), Zinc chloride (20 gms) and Crotonic anhydride (200 gms), 2,6,6-trimethyl-1-cyclohexene (100 ml) is added over a period of 45 minutes at 100° C., after the completion of addition, the mixture is cooled to room temperature and then sodium carbonate aqueous solution (500 ml of 5%) is poured into the mixture. The n-heptane layer containing the product (II) is then separated and washed with water to make the layer neutral. Drying the organic layer over Sodium sulphate, followed by filtration and distillation under atmospheric pressure to obtain the crude product which is further fractionally distilled at about 90° C. and under a reduce pressure of 5 mm Hg to get 1-(2,6,6-trimethyl-2-cyclohexene-1-yl)-but-2-ene-1-one (II) commonly known as α-Damascone, having similar properties given in Example 1, in the yield of 75 gms and having a purity of 95% (GLC).

### EXAMPLE 3

### Preparation of 1-(2,6,6-trimethyl-2-cyclohexene-1-yl)-but-2-ene-1-one from 2,6,6-trimethyl-1-cyclohexene

To a stirred suspension of n-heptane (200 ml), Zinc chloride (30 gms) and Crotonic anhydride (200 gms), 2,6,6-trimethyl-1-cyclohexene (100 ml) is added over a period of 45 minutes at 100° C., after the completion of addition, the mixture is cooled to room temperature and then sodium carbonate aqueous solution (500 ml of 5%) is poured into the mixture. The n-Heptane layer containing the product (II) is then separated and washed with water to make the layer neutral. Drying the organic layer over Sodium sulphate, followed by filtration and distillation under atmospheric pressure to obtain the crude product which is further fractionally distilled at about 90° C. and under a reduce pressure of 5 mm Hg to get 1-(2,6,6-trimethyl-2-cyclohexene-1-yl)-but-2-ene-1-one (II) commonly known as α-Damascone, having similar properties given in Example 1, in the yield of 85 gms and having a purity of 95% (GLC).

Advantages

1. α-Damascone has a strong rose odor with a pronounced green, fruity undertone reminiscent of green apples.

2. α-Damascone highly effective in enriching odor of natural as well as artificial aromas.

3. The present process has applications in the fields of flavor, perfumery & organic chemical synthesis.

4. The present process is easy to perform and economical.

1. A process for the preparation of α-Damascone [1-(2,6,6-trimethyl-2-cyclohexene-1-yl)-2-buten-1-one] represented by formula (II) 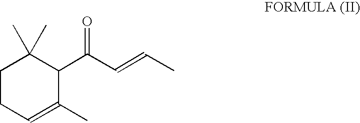
said process comprising the steps of:
(a) adding 2,6,6-trimethyl-1-cyclohexene over a period of 30-95 minutes to a refluxing mixture of an hydrocarbon solvent, Lewis acid and Crotonic anhydride at a temperature in the range of 60-120° C.;
(b) cooling the reaction mixture of step (a) to an ambient temperature of 25-30° C.;
(c) pouring the cooled reaction mixture of step (b) into aqueous alkali carbonate solution and stirring the reaction mixture for about 5-20 minutes;
(d) separating the organic layer and washing the organic layer of step (c) with water till the washing becomes neutral;
(e) drying and removing organic solvent from the organic layer to obtain crude product and purifying the crude product to obtain pure α-Damascone of formula (II).

2. A process according to claim 1, wherein the hydrocarbon solvent is selected from the group consisting of n-hexane, n-pentane, n-propane, n-heptane, cyclopentane or cyclohexane and most preferably cyclohexane or n-heptane.

3. A process according to claim 1, wherein the Lewis acid is selected from the group consisting of aluminum chloride, aluminiumbromide, zinc chloride, zinc bromide or borontrifluoride etherate and most preferably zinc chloride.

4. A process according to claim 1, wherein the alkali carbonate is selected from the group consisting of sodium bicarbonate, potassium bicarbonate, sodium carbonate or potassiumcarbonate and most preferably sodium carbonate.

5. A process according to claim 1, wherein the strength of aqueous alkali carbonate solution is about 5%.

6. A process according to claim 1, wherein the drying of organic layer is done by passing over anhydrous sodium sulphate.

7. A process according to claim 1, wherein the crude product is subjected to fractional distillation at a temperature of about 90° C. and at a pressure of about 5 mm.

8. A process according to claim 1, wherein the crude product is purified by way of vacuum distillation.

9. A process according to claim 1, wherein the product α-Damascone is obtained in the yield of 70%-85% having a purity of more than 95% (GLC).

10. A process according to claim 1, wherein the acylation reaction can also be performed in the absence of solvent at about 20-60° C.

11. A process according to claim 1, wherein the weight ratio of the organic solvent: 2,6,6-Trimethyl-1-Cyclohexene is about 1:3 to obtain higher yields, preferably 1.2-1.5 times the weight of starting material (I).
